(19) United States
(12) Patent Application Publication
(10) Pub. No.: US 2003/0078487 A1
(43) Pub. Date: Apr. 24, 2003

(54) Ocular pressure measuring device

(76) Inventors: Robert E. Jeffries, Rogers, AR (US); Lee Birchansky, Cedar Rapids, IA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/216,418

(22) Filed: Aug. 9, 2002

Related U.S. Application Data
(60) Provisional application No. 60/311,137, filed on Aug. 9, 2001.

(51) Int. Cl. A61B 3/16

(52) U.S. Cl. 600/398

(57) ABSTRACT

An intraocular pressure measuring device including a pressure sensor that is dimensioned to be placed in a cornea or sclera. An intraocular pressure measuring system that includes a pressure sensor positioned in a cornea or sclera and an external device outside the eye, the external device wirelessly communicating with the pressure sensor. A method for measuring intraocular pressure that includes inserting a pressure sensor in a cornea or sclera and sensing intraocular pressure. An ophthalmic device that includes an exoplant and an intraocular pressure sensor connected to the exoplant. An ophthalmic instrument that includes a device adapted to wirelessly interrogate a medical apparatus implanted in the cornea or sclera. A propagating signal for determining intraocular pressure is also described.

### RELATED APPLICATION

This application claims priority under 35 U.S.C. 119(e) from U.S. Provisional Application Serial No. 60/311,137, filed Aug. 9, 2001, which application is incorporated herein by reference.

U.S. Pat. No. 6,193,656, titled “Intraocular Pressure Monitoring/Measuring Apparatus and Method”, issued Feb. 27, 2001 and having the same inventors as the present application, is incorporated by reference in its entirety for any purpose.

WIPO publication WO 00/45693, titled “Intraocular Pressure Monitoring/Measuring Apparatus and Method”, published Aug. 10, 2000 and having the same inventors as the present application, is incorporated by reference in its entirety for any purpose.

### FIELD OF THE INVENTION

The present invention relates to an intraocular pressure measuring/monitoring apparatus and method thereof.

### BACKGROUND OF THE INVENTION

The American Academy of Ophthalmology has reported that about two million people in the United States have primary open angle glaucoma (the most common of several types of glaucoma). About seven million office visits are made each year by people with glaucoma or those suspected of having glaucoma. Glaucoma is the second leading cause of legal blindness in the United States and the leading cause of legal blindness in African-Americans. About 80,000 people in the United States alone are legally blind from glaucoma, not counting those with lesser visual impairment.

By definition, glaucoma is a group of eye diseases characterized by an increase in intraocular pressure which causes pathological changes in the optic disc and nerve fiber layer of the retina with resultant typical defects in the field of vision. The relationship between glaucoma and intraocular pressure is fundamental to proper treatment planning for glaucoma.

Normal intraocular pressure is considered to be less than 22 mm Hg. Intraocular pressure is measured above the atmospheric pressure. However, at least one in six patients with glaucoma may have pressure below this normal level, e.g. in the range of about 10-21 mm Hg, and yet still have progressive eye damage. Thus, it is important to follow an individual intraocular pressure trend over a period of time to determine if their pressure is increasing relative to their individual pressure history. Also, at any single test, about one half of all glaucoma patients will exhibit measured intraocular pressures below 22 mm Hg but actually will have average intraocular pressures higher than 22 mm Hg. This makes frequent testing necessary to obtain an accurate assessment of a patient's average intraocular pressure.

Most current methods of routine intraocular pressure measurements rely on applanating a plunger against the cornea. The degree to which a portion of the cornea is deformed indicates the pressure inside the eye resisting this deformation. All of these methods are inferring the intraocular pressure rather than measuring it directly. Some specialists believe that the thickness of the cornea can vary from person to person, and that other factors such as corneal scars or previous surgery may affect the accuracy of these measurements. Also, most of these methods require that topical anesthesia be placed on the cornea prior to measuring the pressure and the measurements be made by trained personnel. Therefore, there is a need to develop techniques to make repeated and/or continuous measurements and to enable persons other than trained personnel to make such measurements.

An example of a method for measuring intraocular pressure is with Goldman applanation tonometry. It is known that with Goldman applanation tonometry, the accurate reading is obtained at a mean corneal thickness of 0.545 mm. It has been shown that for every 0.070 mm variation in central corneal thickness, intraocular pressure is overestimated or underestimated by 5 mmHg. As a result, through the use of Goldman's method, many pressure readings in glaucoma patients may be overestimated, and many patients with genuinely high intraocular pressure may be missed. As more data become available, an increasing number of ophthalmologists are questioning the validity of readings obtained through use of the Goldman applanation tonometer. Accordingly, there is a need for a more precise method of measuring intraocular pressure. Moreover, due to the increasing popularity of vision correction surgeries, such as photorefractive keratotomy (PRK) and laser in situ keratomileusis (LASIK), greater numbers of people will have thinner corneas. Thinner corneas may result in inaccurate intraocular pressure readings. As a result, eye disease such as glaucoma may not be accurately diagnosed.

U.S. Pat. No. 5,005,577 discloses an intraocular lens pressure monitoring device based on radiosonde technology. Radiosonde technology has been around for decades. It is unclear from the '577 patent how to make or use such an intraocular pressure monitoring device to carry out the invention. Specifically, the technology disclosed in the '577 patent has not been miniaturized in such a way to make it possible to insert into the eye.

Further, the '577 patent discloses an intraocular lens pressure monitoring device as a part of an integrated intraocular lens system, not a stand-alone device. If replacement of the monitoring device is needed, it would be difficult to separate the device from the lens without major surgery.

The '577 patent also discloses active sensors. An active sensor usually includes a power supply and a transmitter. As indicated in the '577 patent, an active sensor is generally too large in size to be implanted in the eye. Although it is speculated that technology will progress to the point to allow an active sensor to be implanted in the eye, the '577 patent does not teach what technology may be used and how it could be used to resolve the above-mentioned problems and addresses the above-mentioned concerns.

Accordingly, there is a need for a miniaturized device capable of inserting into an eye to monitor/measure intraocular pressure accurately, frequently, and continuously. There is also a need for a stand-alone intraocular pressure monitoring/measuring device separate from an intraocular lens system.

### SUMMARY OF THE INVENTION

The present invention includes a device for measuring intraocular pressure, which includes a pressure sensor that is dimensioned to be placed in the cornea, sclera, and/or limbus. In an embodiment, the pressure sensor is positioned in the cornea. In an embodiment, the pressure sensor is positioned in the sclera. In an embodiment, the pressure sensor is positioned beneath the outer surface of the eye and not in contact with either the aqueous humor or the vitreous humor.

The pressure sensor of the present invention is miniaturized such that it is implantable in the corneosclera without interfering with vision. An embodiment of the pressure sensor is a micro electromechanical machined system (MEMS). An embodiment of the pressure sensor is a passive, resonant transducer. An embodiment of the pressure sensor is a silicon resonant transducer. An embodiment of the pressure sensor is a polysilicon resonant transducer. An embodiment of the pressure sensor includes an integrated circuit. An embodiment of the pressure sensor is a sensor fabricated using very or ultra large scale manufacturing techniques. An embodiment of the pressure sensor includes fabricating same from biologically inert materials and/or encasing the pressure sensor in a biologically inert casing. An embodiment of the pressure sensor has an area of about one to two square millimeters and a thickness of less than about the thickness of the cornea. An embodiment of the pressure sensor has a first dimension of about 1 millimeter. An embodiment of the pressure sensor has a second dimension in a range of about 200-300 microns. An embodiment of the pressure sensor has a second dimension of about 100 microns.

The present invention further includes a system for measuring intraocular pressure, including a pressure sensor and a device external to the eye. The device wirelessly communicates with the pressure sensor. In an embodiment, the device includes an energy source for reading data, including pressure data, from a pressure sensor. An embodiment of the device includes a calibration unit that calibrates a reading from the pressure sensor to correct for thickness of the cornea, sclera, or limbus. In an embodiment, the external device includes a calculation unit that subtracts atmospheric pressure from a pressure reading from the pressure sensor to determine intraocular pressure.

The present invention further includes a method for measuring intraocular pressure. The method includes inserting a pressure sensor in the cornea, sclera or limbus and sensing intraocular pressure with the pressure sensor. An embodiment includes inserting the pressure sensor in the cornea. An embodiment includes inserting the pressure sensor in the sclera.

The present invention further includes an ophthalmic device including an implant and an intraocular pressure sensor connected to the implant. In an embodiment, the implant is a vision correction implant such as a corneal ring.

The present invention further includes an ophthalmic instrument, including a device external to an eye and adapted to wirelessly interrogate a medical apparatus implanted in the corneosclera.

The present invention further includes a propagating signal including a first signal traveling through a corneosclera to excite a pressure transducer and a second signal produced by the pressure transducer due to excitation by the first signal. In an embodiment, the second signal includes intraocular pressure data and travels back through the corneosclera. In an embodiment, the first signal travels through less than the full thickness of the corneosclera before exciting the pressure transducer. In an embodiment, the second signal travels through less than the full thickness of the corneosclera before exiting the corneosclera. In an embodiment, the second signal is received by an external device and the intraocular pressure data is converted into pressure units. In an embodiment, the first signal is produced by the same external device as the second signal. In an embodiment, the second signal travels through the air after between the corneosclera and the external device. In an embodiment, both the first signal and the second signal are light signals. In an embodiment, both the first signal and the second signal are optical signals.

### BRIEF DESCRIPTION OF THE DRAWINGS

FIG. 1 is an elevational view of an eye for which the present invention can be adapted.

FIG. 2 is a partial cross-sectional view of the FIG. 1 eye.

FIG. 3 is a plan view showing one step in a method for inserting the present invention into an eye.

FIG. 4 is a partial cross-sectional view of FIG. 3.

FIG. 5 is a plan view showing one step in a method for inserting the present invention into an eye.

FIG. 6 is a cross-sectional view of FIG. 5.

FIG. 7 is a plan view showing one step in a method for inserting the present invention into an eye.

FIG. 8 is an elevational view of an eye with the present invention.

FIG. 9 is a view of the system according to the present invention with an eye shown in partial cross-section.

FIG. 10 is a view of a device of the present invention external to the eye.

FIG. 11 is a cross-sectional view of a pressure sensor according to an embodiment of the present invention.

FIG. 12 is a view of a combination corneal ring and pressure sensor according to an embodiment of the present invention.

FIG. 13 is a view of a method according to the present invention.

FIGS. 14A through 14D are views of an implant assembly according to the present invention.

FIG. 15 is a view of an embodiment of an implant according to the teachings of the present invention.

### DETAILED DESCRIPTION

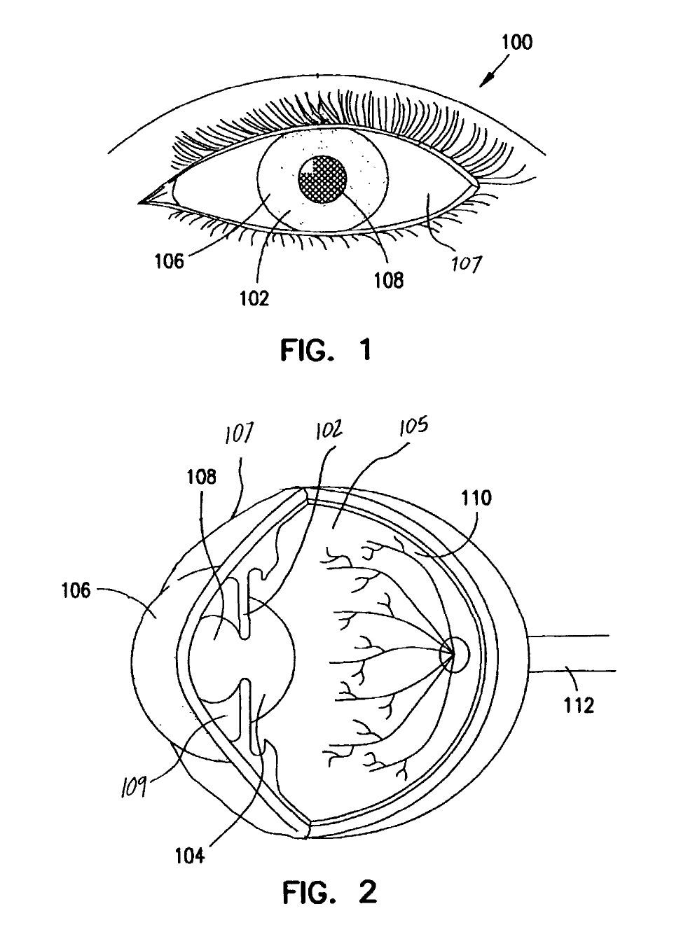

FIGS. 1 and 2 illustrate the major components of an eye 100 including an iris 102, a natural crystalline lens 104, vitreous humor 105, a cornea 106, a sclera 107, a pupil 108, aqueous humor 109, a retina 110, and an optic nerve 112. The present invention is generally directed to an ocular pressure measuring device capable of insertion into the eye 100. The device includes a miniature pressure sensor for insertion into the eye. It will be recognized that insertion into the eye includes any placement of the sensor beneath a surface of the eye. In an embodiment, the sensor is inserted into the cornea 106. In an embodiment, the sensor is inserted in to the sclera 107. In an embodiment, the sensor is inserted into the limbus.

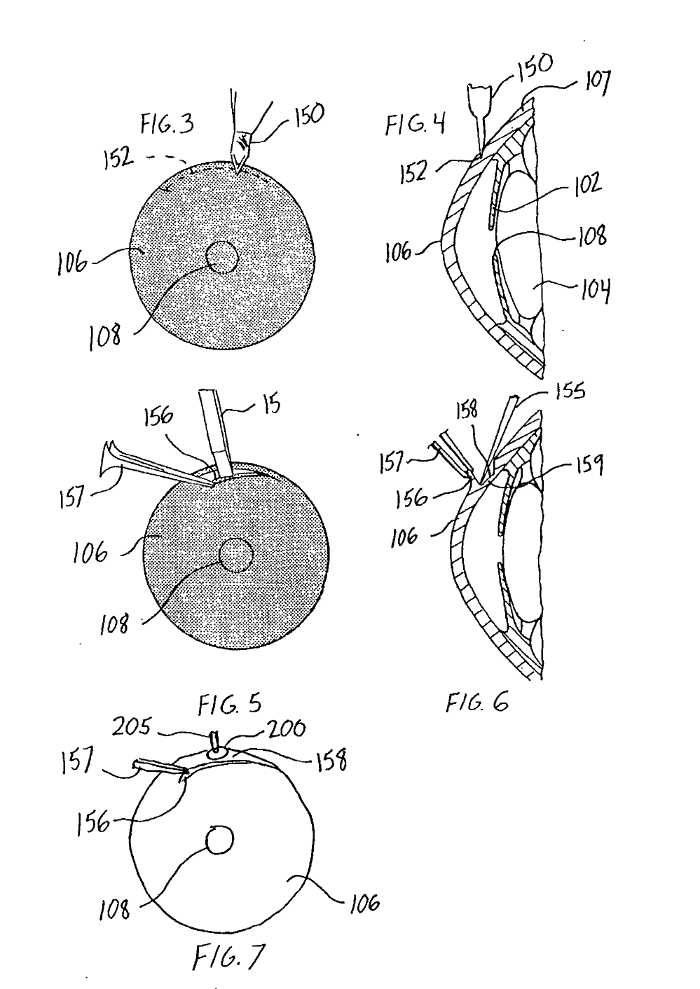

FIGS. 3 and 4 illustrate a first step of one embodiment of a method according to the present invention. A tome, knife or scalpel 150 creates an incision 152 in cornea 106. In an embodiment, the incision 152 extends about half the thickness of the cornea 106. Typical cornea thickness range from about 410 microns to about 710 microns. In an embodiment, the incision 152 is positioned radially outwardly from the pupil 108 such that the incision is not in the line of sight. In an embodiment, the incision 152 is made essentially at the boundary between the cornea 106 and the sclera 107. In an embodiment, the incision is made in the sclera 107 adjacent the cornea. It is desirable to keep the incision out of the line of sight so that patient's vision is not impaired by the incision.

FIGS. 5 and 6 illustrate another step of the present method. A further tome, knife or scalpel 155 is used at the incision to create a flap 156, which in the illustrated embodiment is held away from the remainder of the eye tissue by forceps or tweezers 157. Flap 156 has a thickness of about 160 microns. In an embodiment, the flap has a thickness of at least 150 microns. This creates a pressure sensor receiving pocket 158 adjacent the incision 152. A residual layer of corneal tissue 159 separates the pocket 158 from the aqueous humor 109. The residual corneal layer 159 will have a thickness of about 275 microns. In an embodiment, the residual corneal layer will have a thickness of at least 270 microns. It is desirable to create the flap 156 and pocket 158 as small as necessary to accommodate the ocular pressure measuring device. Consequently, if the ocular pressure sensor has a first dimension, e.g., diameter or length, of about 1 millimeter, then the incision 152 has a length of over 1 millimeter, e.g., at least two millimeters and/or less than four millimeters. The pocket 158 is sized to be greater than the ocular pressure sensor. In an embodiment, the pocket has a depth greater than the height of the ocular pressure sensor. In an embodiment, the pocket 158 has a surface area greater than the ocular pressure sensor. In an embodiment, the pocket 158 has a volume greater than the ocular pressure sensor. In an embodiment, the pressure sensor has a surface area of about one to two millimeters square. In an embodiment, the pressure sensor has a thickness less than about the thickness of the cornea. The thickness of the cornea, in an embodiment, is measured at the area of the incision where the pressure sensor is implanted. In another embodiment, the pressure sensor has a thickness of about less than the thickness of the sclera. The thickness of the sclera, in an embodiment, is measured at the area of the incision where the pressure sensor is implanted.

It will be appreciated by one of ordinary skill that LASIK procedures may be adapted to insert the pressure sensor 200 into the cornea, sclera, and/or limbus. One technique according to the present invention is to create the flap according to LASIK procedures and implant the pressure sensor in the void beneath the flap. The pressure sensor, in an embodiment, is inserted after the ablation of corneal tissue to correct eye focus in a LASIK procedure. Accordingly, the patient who is already undergoing a medical procedure on her eyes also receives an intraocular pressure sensor in a single procedure.

It will also be appreciated that the pressure sensor 200 could be attached to the flap 156, and then the flap is moved back over the pocket 158. Thus, the pressure sensor is implanted in the outer surface of the eye.

FIG. 7 illustrates an ocular pressure sensor 200 being help by forceps 205 and being inserted into pocket 158. Once the sensor 200 is inside the pocket 158, the physician returns the flap over the sensor 200 and the pocket 158. In an embodiment, the flap 156 is lightly brushed to smooth the surface of the eye as much as possible and remove as much air within the pocket 158 as possible. The corneal flap 156 heals substantially similar to that described in the literature regarding LASIK and INTACS® surgical procedures.

Other eye surgery techniques are described in U.S. Pat. Nos. 6,258,110; 6,175,754; 6,079,417; 5,846,265; and 5,599,341, which are incorporated herein by reference. Such eye surgery techniques may be adapted, at least in part, for use with an embodiment of the present invention. The present description describes a scalpel as the device for creating the flap. In some embodiments, the devices described in U.S. Pat. Nos. 6,254,619; 6,165,189; 6,132,446; and 6,126,668, which are incorporated herein be reference, may be adapted for use with the present invention.

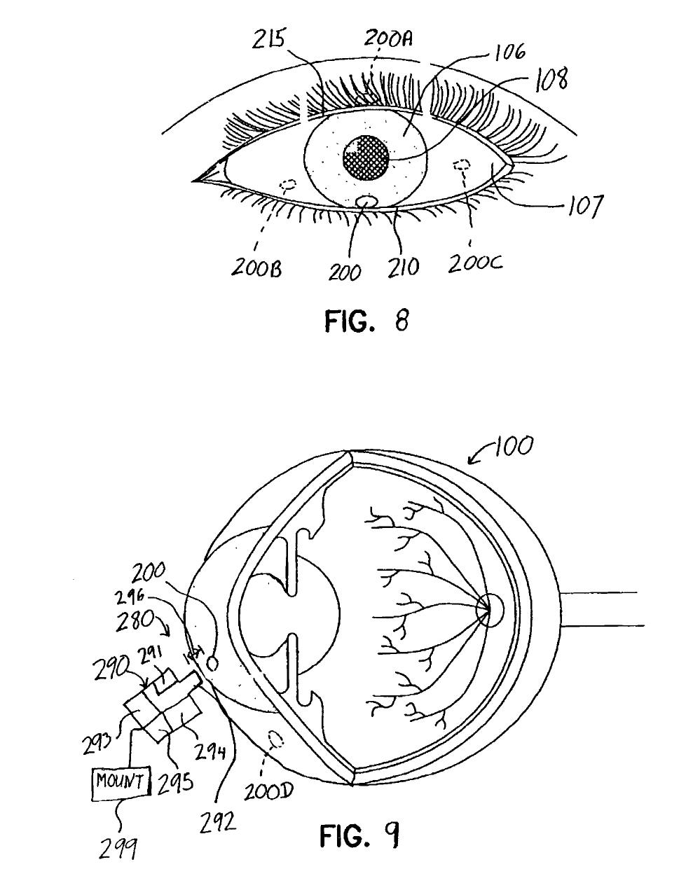

The above described embodiments address inserting the pressure sensor 200 in the periphery of cornea 106. The periphery is generally defined as the area of the cornea outside the area of the pupil. It is desired to position the sensor 200 outside the pupil so as to not interfere with the patient's vision. In an embodiment, the corneal periphery is outside the largest diameter of the pupil, i.e., pupil size in a very low light environment. In an embodiment, sensor 200 is implanted essentially in the twelve o'clock position in the periphery of cornea 106 above the pupil. It is intended that such a positioning aid in the taking of readings from the sensor 200. In an embodiment shown in FIG. 8, the pressure sensor 200 is positioned in the corneal periphery at the six o'clock position beneath the pupil 108. The pressure sensor 200 is shown at least partially above the lower eye lid 210 such that the sensor 200 is visible. In another embodiment shown in FIG. 8, pressure sensor 200A is positioned beneath the upper eyelid 215. The present description uses like reference numbers, sometimes with alphabetical suffixes, for like elements. Accordingly, pressure sensors 200, 200A, 200B, 200C, 200D, 200E, and 200F indicate substantially similar elements. It will be appreciated that sensors 200, 200A, 200B, 200C and 200D are identical except for being in different positions as described herein. The pressure sensor 200A is not visible based on its positioning unless the eyelid 215 is lifted further than its normal open state. In an embodiment, the pressure sensor 200A is positioned in the cornea 106. Such a position of the sensor 200A depends on the structure of the patient's body including the eye and eyelid. In an embodiment, pressure sensor 200A is positioned beneath the eye surface at least partly in the cornea. In an embodiment, the pressure sensor 200A is positioned at least partially in the sclera 107. In an embodiment, the pressure sensor 200A is positioned completely in the sclera 107 beneath the eyelid 215.

It is within the scope of the present invention to also position an ocular pressure sensor 200B and 200C in the sclera 107. Similar procedures to those described herein with regard to positioning an ocular pressure sensor in the cornea are adapted to position an ocular pressure sensor in the sclera. The sensor 200B is shown adjacent the lower eyelid 210 toward the outer corner of the eye. Sensor 200C is shown in a three o'clock position, essentially inwardly of the pupil 108. In another embodiment, sensor 200C is aligned between the pupil and the inside corner of the eye to aid in its location for taking measurements. The sensor 200 may also be positioned in the cornea 106 relative to other landmarks of eye such as the inner corner and the outer corner. If a standard is adopted by the medical community or a specific medical practice, then taking readings will follow the same procedure for each patient. That is, the sensor 200 is in the same position for all patient's unless a medical reason exists for implanting the sensor 200 in another position.

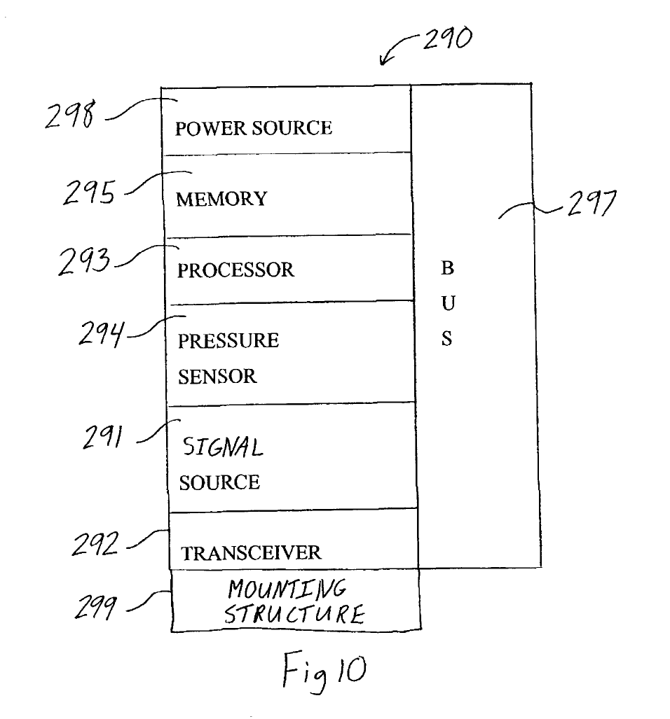

FIG. 9 illustrates a system 280 for measuring intraocular pressure, including the miniature pressure sensor 200 and an external device 290. External device 290 is outside the outer surface of the corneosclera 106 and 107 and is adapted to wirelessly communicate with the pressure device 200. The external device 290 includes an energy source 291 for supplying energy to the pressure sensor 200, which energy causes the pressure sensor to supply the external device 290 with data regarding the intraocular pressure (FIGS. 9 and 10). In an embodiment, the energy source 291 is a light source. In an embodiment, the energy source 291 is a laser. In an embodiment, the laser is unmodulated light source. In an embodiment, the energy source 291 is a radio-frequency source. In an embodiment, the energy source 291 is an electromagnetic source. In an embodiment, the energy source 291 is a acoustic source, sound wave source or sonar source. The energy source 291 includes a transceiver 292, which is adapted to transmit energy from energy source 291 to the pressure sensor 200, which is a passive device in an embodiment. The transceiver 292 receives a signal back from the pressure sensor 200. Embedded in the signal is the intraocular pressure data. In an embodiment, energy source is a light source and transceiver 292 includes a fiber optic and a light detector.

The external device 290, in an embodiment, includes an environmental pressure sensor 294 that measures the atmospheric or environmental pressure experienced by the eye. It is known that atmospheric pressures influence intraocular pressure measurements.

An embodiment of the external device 290 includes computational circuitry 293 such as a processor, application specific integrated circuits, arithmetic logic units, solid state electronics and the like. The circuitry 293 controls operation of the external device and is adapted to compute the intraocular pressure. The circuitry 293 receives the intraocular pressure data, which is relative to the environmental pressure, from the sensor 200 and environmental pressure data from the sensor 294. Circuitry 293 subtracts the environmental pressure data from the intraocular pressure measurement resulting in an intraocular pressure reading. The normal human eye has an intraocular pressure of 10-20 mm Hg above the atmospheric pressure. A higher pressure may be an indication of glaucoma. Moreover, a trend of increased intraocular pressure may be an indication of glaucoma.

In an embodiment of the present invention, the computational circuitry 293 may also correct for the effect of corneal thickness on intraocular pressure measurements and transmission of signals between the external device 290 and pressure sensor 200. Corneal thickness can be measured using techniques known to those of skill in the art. An example of such a technique is described in U.S. Pat. No. 6,193,371, titled “Keratometer/Pachymeter”, herein incorporated by reference. It is believed that the thickness of the residual corneal layer 159 between the pressure sensor and the aqueous humor 109 may influence the readings of the pressure sensor 200. In the embodiment of a MEMS-based pressure sensor 200 the residual corneal layer 159 may reduce or increase the strain on the pressure sensor 200 and alter its pressure measurement. Accordingly, such an alteration can be measures based on the pressure sensor 200 and programmed into the external device 290 to correct for these alterations.

An embodiment of the external device 290 includes a memory 295 adapted to store data from the pressure sensor. Memory 295 also may store pressure data as computed by the computational circuitry 293.

An embodiment of the external device further includes a bus 297 that electrically connects and communicates data signals, control signals, and power between the memory 295, the computational circuitry 293, the energy source 291, environmental pressure sensor 294, and a power source 298. Power source 298 may be a portable power source such as batteries, rechargeable and non-rechargeable. The power source 298 may also be a circuit for connecting the device 290 to a power grid, for example through a common electrical outlet. Power source 298 may further include a transformer for converting the power to a level acceptable to the rest of the device 290.

It is desirable to reduce the distance, indicated at 296 in FIG. 9, between the external device 290 and the pressure sensor 200 in an attempt to reduce alignment problems and/or reduce the power of the energy source 291. The present invention implants the pressure sensor 200 in the corneosclera 106 and 107. The thickness of the cornea ranges typically from 410 microns to 710 microns. Thus, when the pressure sensor is mounted in the cornea it is less than about 410 microns to 710 microns from the outer surface of the eye. The transceiver 292 of the external device 290 is positioned essentially at the outer surface of the eye. Accordingly, the transceiver 292 is slightly greater than 410 microns to 710 microns from the pressure sensor 200 in an embodiment of the present invention. In an embodiment, the transceiver 292 is less than about 500 microns from the pressure sensor 200. In an embodiment, the transceiver 292 is less than about 300 microns from the pressure sensor 200. In an embodiment, the transceiver 292 is less than about 200 microns from the pressure sensor 200. In an embodiment, the transceiver 292 is positioned about 250 microns to about 1 millimeter from the pressure sensor 200. In an embodiment, the transceiver 292 is less than 5 millimeters from the pressure sensor. In an embodiment, the transceiver 292 is less than about 2 millimeters from the pressure sensor.

The distance between the transceiver 292 and the pressure sensor 200 is one factor in determining the amount of power necessary to send a signal to activate sensor 200 to measure intraocular pressure. Thus, if the distance is increased, then greater signal power is needed. However, the power level of a signal will be kept below a threshold at which eye tissue may be damaged. Another factor in the amount of power is the transmission media between the transceiver 292 and the pressure sensor 200. In an embodiment, the transmission media includes air and corneal tissue, sclera tissue, and/or limbus tissue. The power of the signal must transmit through the media and communicate with sensor 200. In an embodiment, the signal must transmit through at least one of the cornea, sclera, limbus, and eyelid. Moreover, different types of signals, e.g., light, electromagnetic, RF, and the like, would require different power levels for their signal to transmit through the media an communicate with the sensor 200. Further, different types of signals may increase the distance at which they can communicate with the sensor 200. For example, it is believed that a light signal from a fiber optic transceiver may be positioned up to about two millimeters from the sensor 200 and communicate with it. It is also believed that RF signals could communicate with sensor 200 at a distance of up to about five millimeters in an embodiment and up to about 8 millimeters in an embodiment. Other embodiments of the present invention may allow communication between external device 290 and sensor 200 at distances up to about several tens of millimeters.

In an embodiment where the pressure sensor 200A is positioned beneath an eyelid, e.g. upper eyelid 215 as shown in FIG. 8, the device 290 has an energy source 291 that produces an energy signal that penetrates the eyelid and the corneal tissue 156 covering the pressure sensor 200A. Thus, the interaction between the pressure sensor 200A and device 290 is the same as described herein except that the energy signal, which causes the sensor 200 to produce intraocular pressure data, must transmit through the eyelid as well as the tissue flap 156.

An embodiment of the present invention includes a mounting structure 299 for holding the external device adjacent the outer surface of the eye aligned with the pressure sensor 200 positioned within the eye. An example of the mounting structure 299 includes a body engaging portion such as arms which engage the patient's head, glasses, headbands which encircle the patient's head and the like. In another embodiment, structure 299 is a handgrip.

FIG. 11 illustrates an embodiment of a MEMS-based pressure sensor 200. The theory for the MEMS-based pressure sensor is discussed in an article by Zook et al., entitled “Optically Excited Self-Resonant Microbeams”, published in Sensors and Actuators A 52 (1996), pages 92-98; and an article by Burns et al., entitled “Sealed-Cavity Resonant Microbeam Pressure Sensor”, published in Sensors and Actuators A 48 (1995), pages 179-186. Briefly, a light beam 300 is directed at the sensor 200. Highly modulated reflected light 305 is produced by an interferometric structure composed of a microresonator 307 and vacuum enclosure 309, both formed in a substrate 311. Substrate 311 includes polysilicon, crystal silicon, single crystal silicon, or other micro-machinable materials. An outer housing or coating 313 encloses the remainder of the pressure sensor to protect the body from the pressure sensor internal materials and structures and/or protect the internal materials and structures from bodily tissue and fluids. The housing 313 may include silicone, acrylic, and PMMA. The microresonator 307 is a microbeam extending into the vacuum enclosure 309. This allows remote fiber-optic excitation and readout of the microbeam resonance frequency for single-point or multi-point sensing. The self-resonant configurations of a MEMS-based pressure sensor, which uses optics or other wirelessly excitation, eliminates physical wiring to the outside and eliminates external circuitry required to maintain microbeam resonance. An embodiment of the MEMS-based pressure sensor is operated by irradiating the MEMS-based pressure sensor with a laser light. The MEMS-based pressure sensor resonates to the light in such a way that is indicative of the pressure of the environment in which it resides. The resonance of the reflected laser light is proportional to the ambient pressure surrounding the device, e.g. within the eye. In an embodiment, non-visible wavelengths of light can be used. The selected frequencies of light will penetrate the tissue of the corneosclera and interact with the device 200 to monitor/measure the intraocular pressure.

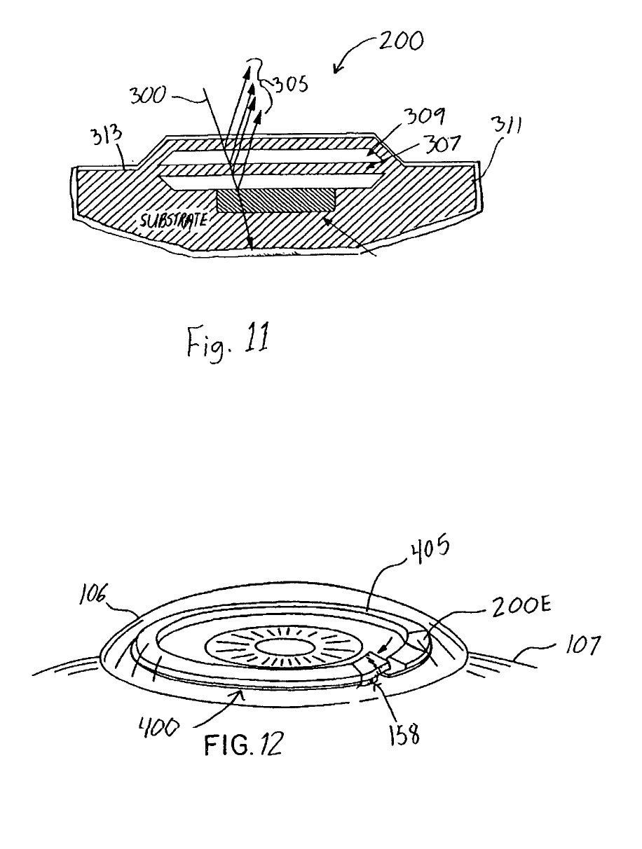

FIG. 12 illustrates an embodiment an assembly 400 of an ophthalmic appliance 405 and pressure sensor 200E. An ophthalmic appliance includes exoplants, ectoplants, and implants. Ophthalmic appliances are discussed in U.S. Pat. Nos. 6,203,513; 4,299,227; and 5,702,414, herein incorporated by reference. In the illustrated embodiment, the appliance 405 is a vision correcting corneal ring implant. Vision correction corneal implants generally alter the shape of the cornea to correct the focus of the eye. Examples of such implants are INTACS® implants which have been sold by Keravision, Inc. of Freemont, Calif. Some of these implants are discussed in U.S. Pat. Nos. 6,214,044; 5,824,086; and 5,645,582, all herein incorporated by reference. Implant 400 includes a vision correction ring 405 and a pressure sensor 200E connected to the ring. In an embodiment, the pressure sensor 200E is integral with ring 405. In an embodiment, the pressure sensor 200E has a cross-sectional area generally equal to ring 405. In a method according to an embodiment of the invention, the pressure sensor 200E is inserted into a same channel through an incision 158 in the cornea as the ring 405. Other types of implants include sclera implants and glaucoma setons. It will be recognized that other embodiments of the assembly 400 include exoplants, such as sclera buckle and the like. An advantage of integrating the pressure sensor according to the present invention with exoplants is the reduction in the number of procedures being performed on the patient. For example, the patient receives a glaucoma seton and a pressure sensor for measuring the intraocular pressure during a same procedure. In an embodiment, the glaucoma seton and pressure sensor are integral. In another embodiment, the exoplant, such as the glaucoma seton, is implanted and the pressure sensor is implanted in the same incision. Consequently, only a single incision made in the patient's body.

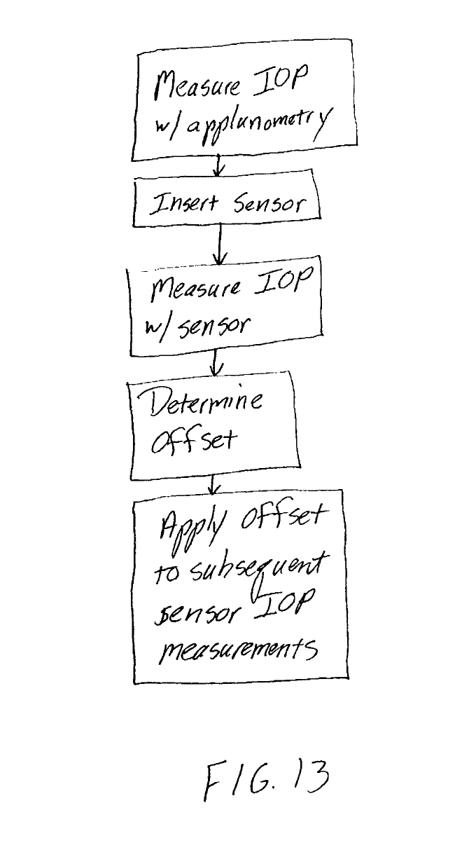

A method for determining the intraocular pressure (IOP) is generally illustrated in FIG. 13. A patient may have prior intraocular pressure reading that were measured using a conventional method such as tonometry or applanometry. The patient's history may be one of a steady IOP reading over time of X mm Hg. When the pressure sensor 200 of the present invention reads IOP it may reveal a pressure of Y mm Hg. Reading X and reading Y may represent the same IOP. However, it is likely that reading X and reading Y are offset by Z mm Hg. This could be due to the pressure sensor 200 measuring IOP relative to a reduced corneal thickness as sensor 200 is in the cornea and measures IOP through only partial corneal thickness. It has been shown that for every 70 micron variation in corneal thickness, IOP may be overestimated or underestimated by 5 mm Hg. Thus, with the sensor 200 positioned 140 microns beneath the corneal surface the IOP may be off by 10 mm Hg. Normal IOP is in the range of 10-21 mm Hg. Consequently, the sensor 200 may measure IOP that is an order of magnitude off the previous tonometry measurements. Thus, sensor 200 may measure IOP which is the same or different than the tonometry measurements. The sensor measurements may or may not indicate a change in the patient's IOP. By determining the offset, for example at the time of implanting the sensor, the offset can be recorded in the patient's records to reduce the likelihood of a misdiagnosis due to a change in the technique and/or structure for measuring IOP.

In an embodiment, the offset is programmed into the external device 290 and stored in memory. The calculation unit may subtract or add the offset from the pressure measurements down loaded from the sensor 200. Thus, there will be no change in IOP data taken using the present sensor compared to prior IOP measurements.

An embodiment of the present invention includes measuring IOP using tonometry, inserting sensor 200 into at least one of the cornea, sclera or limbus, taking an IOP using sensor 200, and determining an offset between the tonometry IOP and the sensor IOP.

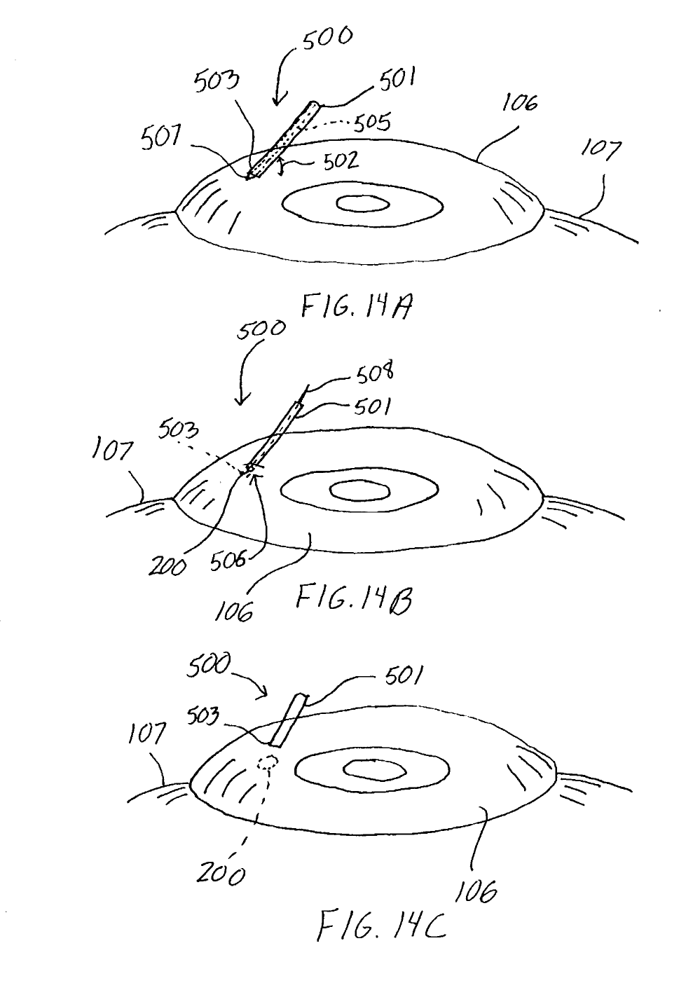

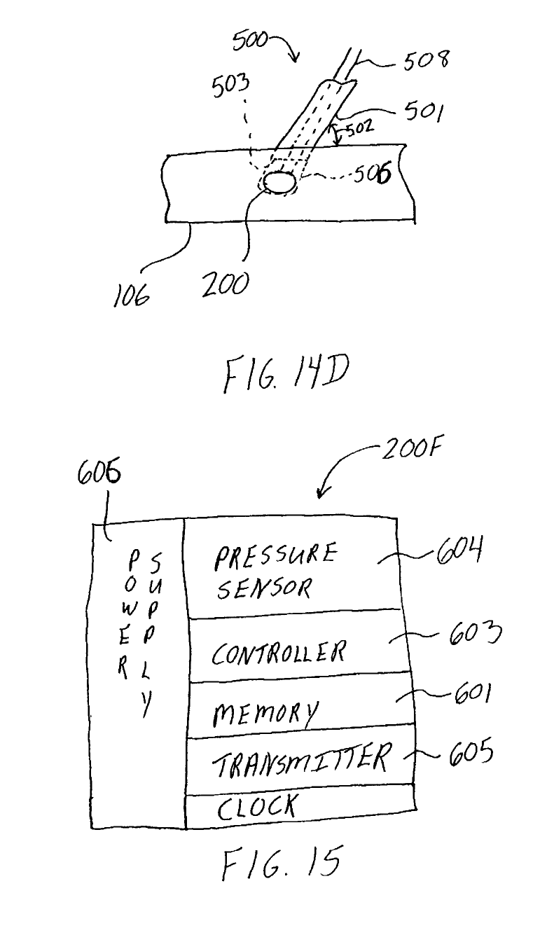

FIGS. 14A-14D illustrate an assembly 500 for inserting the pressure sensor 200 into a cornea 106. The present description will describe implanting sensor 200 into the cornea 106 for the sake of simplicity. It will be understood that an embodiment of the present invention includes implanting sensor 200 into the sclera and/or limbus using a substantially similar assembly 500 and method. The assembly 500 includes a tubular catheter 501 having a distal end 503 adapted to contact the corneal tissue (FIG. 14A). In an embodiment, the catheter 501 is a rigid, bio-compatible tube. A needle 505 extends through the interior of catheter 501 and, in an embodiment, is fixed relative to the catheter. Assembly 500 is held at a non-normal angle 502 relative to the contact area of the cornea 106 at which the assembly contacts the cornea 106. In an embodiment, the angle 502 is less than about 45 degrees. In an embodiment, the angle 502 is less than about 30 degrees. In an embodiment, the angle 502 is less than about 20 degrees. In an embodiment, the assembly 500 creates angle 502 with a contact area of the cornea radially outwardly of the pupil. In an embodiment, the assembly 500 creates angle 502 with a contact area of the outer periphery of the cornea. Needle 505 includes a sharp point 507 for puncturing the eye tissue, in particular the corneal tissue. Point 507 extends beyond catheter distal end 503 by a predetermined distance generally equal to the depth at which the IOP sensor 200 will be implanted into the corneal tissue. Thus, the catheter distal end 503 acts as a depth guide or stop for penetrating the corneal tissue with point 507. The point 507 punctures the corneal tissue to create a pocket 506 adapted for receiving IOP sensor 200. In an embodiment, the implant depth is about 160 microns. In an embodiment, the implant depth is about 250 microns. In an embodiment, the implant depth is less than a range of about 400 microns to about 700 microns. In an embodiment, the needle 505 is retracted proximally from the catheter 501. An implant tool 508 having a distal end releasably contacting the IOP sensor 200 is inserted into the catheter 501 (FIGS. 14B and 14D.). Insertion tool 508 pushes IOP sensor 200 through the interior of the catheter 501 into pocket 506. Insertion tool 508 and catheter 501 are removed from the patient's eye (FIG. 14C). IOP sensor 200 remains in pocket 506 in the cornea 106. In an embodiment, insertion tool 508 is a syringe. In an embodiment, syringe 508 mechanically pushes IOP sensor 200 to catheter distal end 503 and forces IOP sensor 200 into pocket 506 by fluid flow. In an embodiment, the insertion tool 508 is a push rod that mechanically pushes IOP sensor 200 through the catheter 501 into pocket 506.

In an embodiment, the point 507 of needle 505 creates a small incision into the corneal tissue, thereby, creating pocket 506. The needle 505 is proximally retracted while the catheter 501 is moved distally into pocket 506. Catheter distal end 503 maintains pocket 506 while IOP sensor 200 is inserted therein.

An embodiment of the present invention includes the IOP sensor 200F having integral circuits therewith (FIG. 15). The integral circuits may include at least one of a memory 601, clock 602, a controller 603, a sensor 604, and a communication circuit 605 for communicating with a device external to the eye. In an embodiment, these integral circuits require a power supply 606. The power supply 606 is onboard the sensor 200F when the sensor is an active, non-passive circuit. Power supply 606 includes at least one of a battery, capacitor or a photocell or a combination thereof. Conventional eye implants having power supplies did not find acceptance in the medical arts. In the case of batteries, harmful, non-biocapatible materials are included in the batteries. Thus, the potential harm to the eye was not worth the risk of a implant deep within the eye. According to the teachings herein, an implant 200F, such as IOP sensor 200, is implanted in the cornea, sclera, and/or limbus. Thus, the implant including a power source is close to the surface of the eye. Any adverse reaction caused by the implant and/or battery component will be visible upon a relatively easy inspection. Moreover, the implant is not very deep within the eye and may be removed in the doctor's office.

The power supply 606, in an embodiment, is a photocell that is powered by ambient light transmitted through the eye tissue. In an embodiment, the implant is positioned in the cornea so that it receives more light than if the implant was in the sclera. In an embodiment, the implant is covered by a sufficiently thin flap of the sclera such that it receives sufficient light to power the implant. In an embodiment, the photocell is connected to an energy storage device, such as a battery or capacitor, to charge the energy storage device with sufficient power to keep the implant functioning during the night.

The implant 200F with an onboard power supply 606 opens up many potentially beneficial applications. One is the memory 601 that stores medical measurements, such as IOP, temperature, and the like. In an embodiment, the memory 601 can store about 100 measurements. In an embodiment, the memory 601 can store about 200 measurements. These measurements may include a date and/or time stamp for each measurement. The stored data is downloaded according to the teachings herein using communication circuit 605. Such a downloading process may be performed once a day or once a week depending on the frequency of measurements and memory capacity.

The implant has a clock 602, which triggers the implant 200F to take a measurement and store the measurement in memory 601 in an embodiment. The controller 603 may be programmable and interconnected to the clock such that the implant may be programmed to take measurements at various frequencies, e.g. every 15 minutes, hourly, daily, weekly, monthly. Short frequency measurements, e.g. on the order of minutes of tens of minutes, may be beneficial when a drug therapy is being administered. The efficacy and/or side effects of the drug as it relates to measurements that can be taken by the implant could be adequately monitored. Moreover, it is known that IOP varies based on circadian rhythms. Thus, implant 200F could be programmed to take the IOP measurement at the same time every day. This would remove any IOP variations based on circadian rhythms from the data.

The present invention includes a device for measuring intraocular pressure, which includes a pressure sensor 200 that is dimensioned to be placed in the cornea 106. In an embodiment, the pressure sensor 200 is mounted in the sclera 107. The pressure sensor 200 is positioned such that it does not interfere with the patient's vision. The miniature pressure sensor is small, e.g., an integrated circuit, and is biocompatible with the eye including the cornea and the sclera. In an embodiment, the pressure sensor 200 is a micro electro-mechanical system (MEMS). In an embodiment, the pressure sensor 200 is a micro resonant transducer. In an embodiment, the pressure sensor 200 is a silicon-based, micro resonant transducer. In an embodiment, the pressure sensor is a polysilicon resonant transducer. In an embodiment, the pressure sensor includes an integrated circuit. In an embodiment, the pressure sensor 200 only contacts the one of the cornea 106 and the sclera 107 and is free of contact with the vitreous humor 105 and the aqueous humor 109. In an embodiment, the pressure sensor 200 has a surface spaced less than about 400 to 700 microns from an outer surface of the surface of the eye, i.e., an outer surface of one or the other of the cornea and the sclera. In an embodiment, the pressure sensor 200 has a surface spaced about 160 microns from the outer surface of the eye. In an embodiment, the pressure sensor 200 has a surface spaced less than about 400 microns from an outer surface of the eye. In an embodiment, the pressure sensor is positioned in the sclera 107 and beneath the eyelid such that the pressure sensor is not visible without lifting the eyelid. In an embodiment, the pressure sensor 200 is positioned in the sclera and includes an element responsive to an energy source. In an embodiment, the element is responsive to one of sound, radio-frequency and electromagnetic waves.

The present invention further includes a system for measuring intraocular pressure. The system comprises a pressure sensor 200 positioned beneath a surface of the eye and an external device 290 outside eye capable of wirelessly communicating with the pressure sensor. In an embodiment, the pressure sensor 200 is positioned in one of a cornea and a sclera without reducing effective vision. In an embodiment, the external device 290 includes a calibration unit that calibrates a reading from the pressure sensor to correct for thickness of the cornea. In an embodiment, the external device includes a calculation unit that subtracts atmospheric pressure from a reading from the pressure sensor to determine intraocular pressure. In an embodiment, the external device includes an energy source for wirelessly interrogating the pressure sensor. In an embodiment, the energy source is a light source. In an embodiment, the energy source is a laser. In an embodiment, the energy source is one of a sound, radio-frequency, and electromagnetic source.

The present invention further includes a method for measuring intraocular pressure. The method comprises inserting a pressure sensor beneath the surface of the eye and sensing intraocular pressure with the pressure sensor. In an embodiment, the pressure sensor is mounted in the cornea. In an embodiment, the pressure sensor is mounted in the sclera. In an embodiment, the method includes non-invasively transmitting data from the pressure sensor to a device external to the eye. In an embodiment, the method includes interpreting data sensed by the pressure device in view of environmental data. In an embodiment, the method includes subtracting atmospheric pressure from the pressure sensed by the pressure sensor. In an embodiment, the method includes reading the atmospheric data in the same environment as sensing intraocular pressure was performed. In an embodiment, the method includes reading atmospheric data and sensing intraocular pressure occur at about the same time. In an embodiment, the method includes transmitting the data in light. In an embodiment, the method includes transmitting the data in visible light. In an embodiment, the method includes creating a flap in one of the cornea and the sclara, inserting the pressure sensor beneath the flap, and closing the flap. In an embodiment, the method includes closing the flap to completely cover the pressure sensor beneath an outer surface of the eye. In an embodiment, the method includes keeping the pressure sensor free from direct contact to the vitreous humor of the eye. In an embodiment, the method includes keeping the pressure sensor free from direct contact to the aqueous humor of the eye.

An embodiment of the present invention provides a passive pressure sensor which is positioned in the eye at a depth from the surface of the eye on the order of 100 microns. This reduces the amount of tissue and bodily fluids between the sensor 200 and the interrogation device 290. Accordingly, it is easier to align the device 290 with the sensor 200. Moreover, the device 290 can activate the sensor 200 using less power.

It is important to follow the trend of a particular patient's IOP. An individual may have glaucoma even when her pressure is within the normal range, e.g., below 22 mm Hg. Such an individual may have an IOP trend that is increasing over time but is still within the normal range. As an example one patient may have had good vision for most of their life with an IOP of about 14 mm Hg. Recently, this patient's IOP has increased to 17 mm Hg. Thus, the IOP trend is increasing. This may be an indication of eye disease such as glaucoma. Further testing and examinations may be needed.

It is believed that the present invention provides improved devices and methods for measuring intraocular pressure. The present invention provides for less invasive testing, e.g. no eye drops and tonometer are needed. This is intended to improve diagnosis of pressure related eye diseases such as glaucoma. A micro-sensor is implantable into the outer layers of the eye, such as the cornea and sclera. It is further intended that embodiments of the present invention be practiced on an out patient basis by reducing the level of invasive surgery over prior pressure sensor implants. That is, some embodiments of the present invention due do not require cutting into or inserting items into the interior of the eye, such as into the aqueous humor, vitreous humor, attaching items to the iris and/or lens. This allows for patient self-monitoring simply by pointing the device 290 at sensor 200 and activating the sensor an IOP measurement is taken. Due to its less invasive nature, the present invention would allow for more frequent monitoring of IOP.

It will be appreciated that the term “corneosclera” refers to both the corneal tissue and the sclera tissue as well as the limbus tissue joining the cornea and sclera. Some embodiments of the present invention require the pressure sensor 200 to be implanted in the cornea. Some embodiments of the present invention require the pressure sensor 200 to be implanted in the sclera. Some embodiments of the present invention allow the pressure sensor 200 to be implanted in the limbus. It will be understood that the techniques described herein that refer to one of the cornea or sclera are adaptable to the other of the cornea or sclera and the limbus and remain within the scope of the present invention.

While the above description discusses certain type of pressure sensors, e.g., solid state, micro pressure transducers, MEMS-based sensors, PRT's, etc., it will be appreciated that other types of suitable transducers and/or pressure sensors can be employed in various embodiments of the present invention without departing from the scope of the present invention.

The aforementioned description is not to be interpreted to exclude other devices advantageously employing the present invention. Other embodiments may be desired by those skilled in the art without departing from the spirit and scope of the present invention. For example, the above procedure for inserting the ocular pressure sensor into the eye is but one embodiment that may be used to insert the sensor. One skilled in the art will recognize variations on the described insertion procedure, which variations are within the intended scope of the present invention.

What is claimed is:

1. A device for measuring intraocular pressure, comprising a pressure sensor that is dimensioned to be placed in one or both of a cornea and a sclera without reducing effective vision, the pressure sensor being bio-compatible with the one or both of the cornea and the sclera.

2. The device of claim 1, wherein the pressure sensor is a micro electromechanical system (MEMS).

3. The device of claim 1, wherein the pressure sensor is a polysilicon resonant transducer.

4. The device of claim 1, wherein the pressure sensor is an integrated circuit.

5. The device of claim 1, wherein the pressure sensor only contacts the one or both of the cornea and the sclera.

6. The device of claim 1, wherein the pressure sensor is free of contact with the vitreous humor or the aqueous humor.

7. The device of claim 1, wherein the pressure sensor has a surface spaced less than about 0.5 millimeter from an outer surface of the one or both of the cornea and the sclera.

8. The device of claim 1, wherein the pressure sensor has a surface spaced less than about 400 microns from an outer surface of the one or both of the cornea and the sclera.

9. The device of claim 1, wherein the pressure sensor is positioned in the sclera and beneath the eyelid such that the pressure sensor is not visible without lifting the eyelid.

10. The device of claim 1, wherein the pressure sensor is positioned in the sclera and includes an element responsive to an energy source.

11. The device of claim 10, wherein the element is responsive to one of sound, radio-frequency and electromagnetic waves.

12. A system for measuring intraocular pressure, comprising:
a pressure sensor positioned in a corneosclera without reducing effective vision; and
an external device outside the corneosclera, the external device capable of wirelessly communicating with the pressure sensor.

13. The system of claim 12, wherein the external device includes a calibration unit that calibrates a reading from the pressure sensor to correct for thickness of the corneosclera.

14. The system of claim 12, wherein the external device includes a calculation unit that subtracts atmospheric pressure from a reading from the pressure sensor to determine intraocular pressure.

15. The system of claim 12, wherein the external device includes an energy source for wirelessly interrogating the pressure sensor.

16. The system of claim 15, wherein the energy source is a light source.

17. The system of claim 15, wherein the energy source is a laser.

18. The system of claim 15, wherein the energy source is one of a sound, radio-frequency, and electromagnetic source.

19. A method for measuring intraocular pressure, comprising:
inserting a pressure sensor in a corneosclera; and
sensing intraocular pressure with the pressure sensor.

20. The method of claim 19, wherein sensing the intraocular pressure includes non-invasively transmitting data from the pressure sensor to a device external to the eye.

21. The method of claim 20, wherein sensing intraocular pressure includes interpreting data sensed by the pressure device in view of environmental data.

22. The method of claim 21, wherein interpreting the data includes subtracting atmospheric pressure from the pressure sensed by the pressure sensor.

23. The method of claim 22, wherein interpreting the data includes reading the atmospheric data in the same environment as sensing intraocular pressure was performed.

24. The method of claim 23, wherein reading atmospheric data and sensing intraocular pressure occur at about the same time.

25. The method of claim 20, wherein non-invasively transmitting data includes transmitting the data in light.

26. The method of claim 25, wherein transmitting the data includes transmitting the data in visible light.

27. The method of claim 19, wherein inserting the pressure sensor includes:
creating a flap in the corneosclera;
inserting the pressure sensor beneath the flap; and
closing the flap.

28. The method of claim 27, wherein closing the flap completely covers the pressure sensor beneath an outer surface of the eye.

29. The method of claim 19, wherein inserting a pressure sensor in the corneosclera includes keeping the pressure sensor free from direct contact to the vitreous humor of the eye.

30. The method of claim 19, wherein inserting a pressure sensor in the corneosclera includes keeping the pressure sensor free from direct contact to the aqueous humor of the eye.

31. The method of claim 19, wherein inserting the pressure sensor includes positioning the pressure sensor less than about 0.5 millimeters from an outer surface of the eye.

32. An ophthalmic device, comprising:
an ophthalmic implant; and
an intraocular pressure sensor connected to the ophthalmic implant.

33. The device of claim 32, wherein the ophthalmic implant includes a corneal ring.

34. The device of claim 33, wherein the corneal ring is a vision correction implant.

35. The device of claim 34, wherein the sensor is integral with the corneal ring.

36. The device of claim 32, wherein the sensor is a MEMS

37. The device of claim 32, wherein both the ophthalmic implant and the sensor are adapted to be implanted into the cornea.

38. An ophthalmic instrument, comprising a device external to an eye and adapted to wirelessly interrogate a medical apparatus implanted in the corneosclera.

39. The instrument of claim 38, wherein the device includes an energy source for wirelessly interrogating a passive medical apparatus.

40. The instrument of claim 39, wherein the device includes an environmental pressure sensor and a calculation unit applies data from the environmental pressure sensor to data from the passive medical apparatus.

41. The instrument of claim 38, wherein the device includes a mounting structure generally fixing the device relative to the medical apparatus.

42. A propagating signal, comprising:
a first signal traveling through a corneosclera to excite a pressure transducer; and
a second signal produced by the pressure transducer due to excitation by the first signal, wherein the second signal includes intraocular pressure data and travels back through the corneosclera,

43. The signal of claim 42, wherein the first signal travels through less than the full thickness of the corneosclera before exciting the pressure transducer.

44. The signal of claim 43, wherein the second signal travels through less than the full thickness of the corneosclera before exiting the corneosclera.

45. The signal of claim 44, wherein the second signal is received by an external device and the intraocular pressure data is converted into pressure units.

46. The signal of claim 44, wherein the second signal travels through the air after between the corneosclera and the external device.

47. The signal of claim 42, wherein both the first signal and the second signal include light signals.

48. The signal of claim 42, wherein both the first signal and the second signal include optical signals.
